(19) United States
(12) Patent Application Publication
(10) Pub. No.: US 2004/0064435 A1
(43) Pub. Date: Apr. 1, 2004

(54) Clinical assessment and diagnostic tool for use with peristaltic pump

(76) Inventors: Ahmad-Maher Moubayed, Mission Viejo, CA (US); David Norman White, San Juan Capistrano, CA (US); Linda Thomas, Laguna Niguel, CA (US); Chuck Grey, Irvine, CA (US); Mehryar Michael Parsee, Los Angeles, CA (US)

Correspondence Address: Kit M. Stetina, STETINA BRUNDA GARRED & BRUCKER, Suite 250, 75 Enterprise, Aliso Viejo, CA, 92656, US

(21) Appl. No.: 10/459,079

(22) Filed: Jun. 11, 2003

Related U.S. Application Data
(63) Continuation-in-part of application No. 10/205,965, filed on Jul. 26, 2002.

(51) Int. Cl. G06F 7/00

(52) U.S. Cl. 707/1

(57) ABSTRACT

A method of storing on a remote storage device protocol information for a drug for administration via a peristaltic pump is disclosed. A communications path between the peristaltic pump and the remote storage device is provided. The protocol information for the drug is entered into the peristaltic pump. The protocol information is transferred from the peristaltic pump to the remote storage device. The protocol information for the drug is stored on the remote storage device. History information may be retrieved from the peristaltic pump. A user request is received requesting retrieval of history information from the peristaltic pump. A pump request is formatted to retrieve history information. The pump request to receive history information is transmitted to the peristaltic pump. The history information is received from the peristaltic pump. The history information can be displayed and/or stored.

### CROSS-REFERENCE TO RELATED APPLICATIONS

This application is a continuation-in-part of U.S. application Ser. No. 10/205,965 filed Jul. 26, 2002, the entire contents of which are incorporated by reference herein.

### STATEMENT RE: FEDERALLY SPONSORED RESEARCH/DEVELOPMENT

Not Applicable

### COMPACT DISK APPENDIX

The specification of the present application includes a compact disc appendix which is submitted herewith and incorporated by reference herein. The compact disc was created on Oct. 25, 2002 and includes a total of four (4) files. The first file, “350-9008B CMS User Manual,” is a User's Manual for the Curlin™ Medical Clinical Management System and has a size of 891 Kbytes. The second file, “Curlin Medical 4000 Peristaltic Pump,” is a User's Manual for the Curlin Medical 4000 Peristaltic Pump and has a size of 2,243 Kbytes. The third file, “History File PCA Boluses UCLA_SN102436,” an exemplary history file used to generate the bolus requested vs. bolus received graph over a given period of time shown in FIG. 12 and has a size of 55 Kbytes. The fourth file, “History File TPN Pressure SN102243,” is an exemplary history file that was used to generate the line pressure graph of FIG. 10 and has a size of 33 Kbytes.

### BACKGROUND OF THE INVENTION

The present invention relates generally to medical infusion pumps and more particularly to a system and method for remotely controlling a peristaltic pump.

Traditionally infusion pumps have been used to deliver medications and fluids to patients by intravenous subcutaneous or Epidural methods, according to a controlled rate and dose schedule. Such infusion or peristaltic pumps are known in the art. Peristaltic pumps may be linear, such as those described in U.S. Pat. No. 2,877,714 (Sorg, et. al), U.S. Pat. No. 4,671,792 (Borsannyi), U.S. Pat. No. 4,893,991 (Canon), rotary, such as those described in U.S. Pat. No. 4,886,431 (Soderquist et al.) and U.S. Pat. No. 3,172,367 (Kling) or curvilinear, such as is described in U.S. Pat. No. 6,164,921 (Moubayed et al.).

The pump is normally programmed by a clinician based on a specific patient prescription. The pump is traditionally programmed through a user interface keypad on the pump.

There have been some efforts in the past to establish capabilities of remotely programming the pump through a modem and transferring data through telephone lines. For example, Mediview, which is currently owned by Baxter, provides remote programming capabilities of the Homerun 6060 pump through a modem and telephone line, It allows the clinician to view, at a remote location, the 6060 pump simulated on a computer monitor with its display and keypad. The clinician can view the display of the remote pump on a computer monitor and can interact with the pump using a mouse and keyboard. Remote programming systems, such as those described above may be difficult to program and do not reduce infusion errors.

Thus, there is a need for a system and method for programming a peristaltic pump which reduces prescription programming errors and subsequently infusion errors. The system should also be easy to program, i.e., should not require significant training by the clinician. Preferably, the system and method also provide for clinical assessment and diagnosis of the pump as it is being used for a particular patient.

### BRIEF SUMMARY OF THE INVENTION

An aspect of the present invention may be regarded as a method of storing on a remote storage device protocol information for a drug for administration via a peristaltic pump. The method provides a communications path between the peristaltic pump and the remote storage device. The protocol information for the drug is entered into the peristaltic pump. The protocol information is transferred from the peristaltic pump to the remote storage device. The protocol information for the drug is stored on the remote storage device.

The protocol information may be stored in a drug library on the remote storage device. The protocol information may be selected from the drug library and sent to the peristaltic pump for administration to a patient. The protocol information may be copied from the drug library to a patient library. The protocol information may be exported from the drug library. The exported protocol information may be sent to another user, for example, via e-mail. The exported protocol information may be imported to the drug library. The protocol information in the drug library may be edited.

The protocol information may be stored in a patient library on the remote storage device. The protocol information may be selected from the patient library and sent to the peristaltic pump for administration to a patient. The protocol information may be exported from the patient library. The exported protocol information may be imported to the patient library. The protocol information in the patient library may be edited.

The protocol information for the drug may include associated warnings and precautions.

The remote storage device is a personal computer, such as a laptop computer. The remote storage device may be a handheld storage device, such as a Personal Digital Assistant (PDA).

A current date/time and/or maintenance date may be entered.

Pump calibration functions may be invoked.

Another aspect of the present invention may be regarded as a method for receiving history information from a peristaltic pump. A user request is received requesting retrieval of history information from the peristaltic pump. A pump request is formatted to retrieve history information. The pump request to receive history information is transmitted to the peristaltic pump. The history information is received from the peristaltic pump. The history information is displayed.

The history information may be all of the history information stored in the peristaltic pump. The history information may be the latest prescription. The history information may be a predefined amount of history information, e.g., four kilobytes. The history information may be printed or exported for e-mail to others.

Another aspect of the present invention is clinical assessment and diagnostics. History information received from a peristaltic pump may be extracted. The information is extracted in response to a request for information. The requested information is then extracted from the history file.

In exemplary embodiments the requested information may be patient side administration set (line pressure) information or bolus request information. In the case of bolus request information, the extracted information may include bolus requests and medication administered in response to bolus requests.

The extracted information may be displayed, saved to a file, printed and/or transmitted, e.g., via e-mail. Saved files can be renamed. The extracted information may be in a graphical format.

### BRIEF DESCRIPTION OF THE DRAWINGS

These as well as other features of the present invention will become more apparent upon reference to the drawings wherein:

FIG. 1A illustrates a first embodiment of the present invention wherein a peristaltic pump is in communication with a laptop computer;

FIG. 1B illustrates a second embodiment of the present invention wherein the peristaltic pump of FIG. 1A is in communication with a handheld computing device;

FIG. 2 illustrates an example screen display showing the major functions of one embodiment of the present invention;

FIGS. 3-9 illustrate exemplary screen displays for performing the various functions available from the display shown in FIG. 2;

FIG. 10 is a graph illustrating line pressure for a pump over a given period of time;

FIG. 11 is an excerpt of a history file used to generate the graph shown in FIG. 10;

FIG. 12 is a graph illustrating bolus requested vs. bolus received over a given period of time; and

FIG. 13 is an excerpt of a history file used to generate the graph shown in FIG. 12.

### DETAILED DESCRIPTION OF THE INVENTION

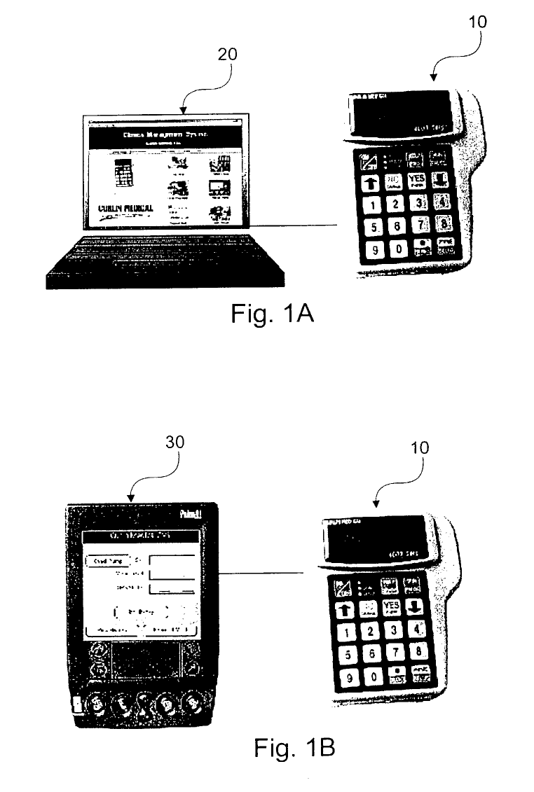

Referring now to the drawings wherein the showings are for purposes of illustrating preferred embodiments of the present invention only, and not for purposes of limiting the same, FIGS. 1A and 1B illustrate a peristaltic pump in communication with a computer capable of programming the pump. The particular pump shown in the Figures is marketed by Curlin™ Medical of Huntington Beach, Calif. and is described in U.S. Letters Pat. No. 6,164,921, the disclosure of which is expressly incorporated herein by reference. However, use of other ambulatory pumps is contemplated herein. Pumps, such as the one shown in FIGS. 1A and 1B are typically stand-alone devices used to administer medication to a patient. The pump 10 shown in FIGS. 1A and 1B can be used as a stand-alone pump. Additionally, the pump shown in FIGS. 1A and 1B can communicate with a computer, such as a laptop computer 20 (shown in FIG. 1A) or a handheld computer, such as a personal digital assistant (PDA) 30 (shown in FIG. 1B).

The present invention helps reduce the risk of prescription programming errors, and thus medication errors, reduces staff costs by providing time-efficient programming of the pump, facilitates remote monitoring of the infusion process, provides an audit trail for billing, validation and archival purposes, and easily integrates into existing systems. The data management functions allow the clinician the ability to create, select, and use protocols and prescriptions, select and transfer personalized prescriptions to a PDA/Palm™ device, provide a “Drug Precautions” page for warnings, indications and instructions, compile data for further analysis, retrieve patient-history files, and generate customized reports from a PC or Palm device.

Preferably, the computing device 20 is a personal computer (PC) with at least a 486 Intel® processor with a system speed of at least 90 MHz (megahertz). In preferred embodiments, the computing device 20 uses a Windows® operating system, such as Windows® 95, 98, ME, 2000 or NT. The computing device 20 should have at least thirty-two (32) MB (megabytes) of random access memory (RAM) and at least eight (8) MB of available storage space. The computing device 20 preferably includes a compact disc read-only memory (CD-ROM) drive. Preferably, the computing device 20 includes a graphics card that is capable of a pixel resolution of 800×600 or better (e.g., super video graphics array (SVGA) or better). In addition to a keyboard, the computing device 20 preferably includes a pointing device, such as a mouse.

The pump shown in FIGS. 1A and 1B, like prior art pumps includes logic (software) for managing the pump.

In exemplary embodiments of the present invention, the computer 20 stores a drug library and a patient library. The drug library stores protocols classified by drug name, programmer name (person who stored the protocol), and creation date. A prescription or protocol can be selected from those stored in the drug library. The prescription can then be sent to the pump attached to the computer. The protocol is then uploaded to the pump.

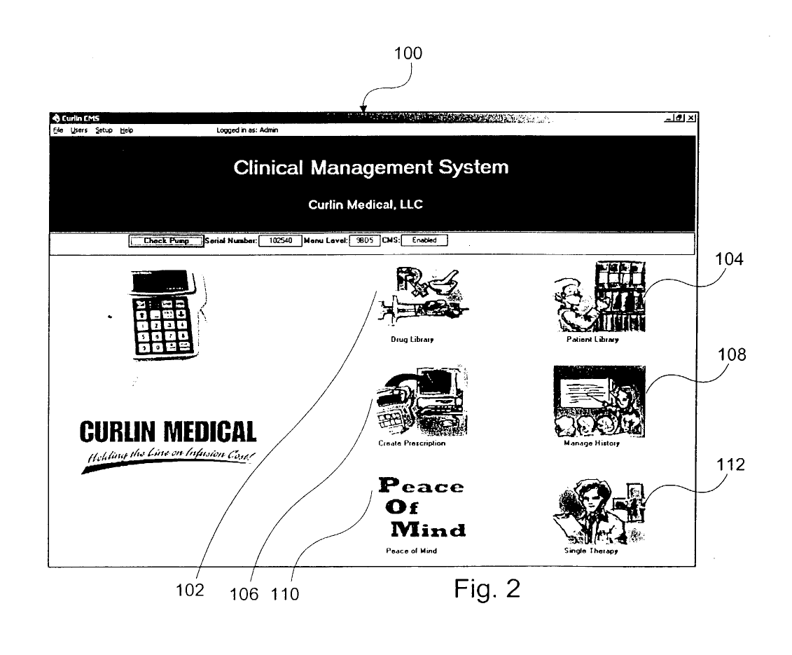

FIG. 2 illustrates an example screen display showing the major functions of one embodiment of the present invention. Preferably, upon starting the program, the user is asked to enter a password. Details in setting up passwords and entering and validating password is not explained in further detail herein as techniques known in the art can be used for security aspects of the present invention including a user login function.

The exemplary screen display 100 shown in FIG. 2 includes controls to access the major functions of the present invention. In the illustrated example, graphical depictions of the function are selected to invoke the various functions. It will be appreciated that other user interface controls, such as menus, could be used to access the functions. The selections available from the main menu shown in FIG. 2 include: Drug Library 102, Patient Library 104, Create Prescription 106, Manage History 108, Peace of Mind 110 and Single Therapy 112. Each of theses functions is briefly summarized next and described in more detail later.

Pressing the Drug Library button 102 invokes the drug library function which allows the user to store and access protocols. Pressing the Patient Library button 102 invokes the patient library function which allows the user to store and access (e.g., copy and export) patient specific prescriptions. Pressing the Create Rx button 104 invokes the create prescription function which allows the user to enter and store information in the drug library or the patient library. Pressing the Manage History button 106 invokes the manage history function which allows the user to download the pump history for archiving, documentation, review or analysis. Pressing the Peace of Mind button 108 invokes the peace of mind function which downloads a recently programmed therapy for documentation, validation or verification. Pressing the Single Therapy button 110 invokes the single therapy function which converts the pump into a PCA, TPN, continuous, intermittent, or variable therapy only pump for manual programming.

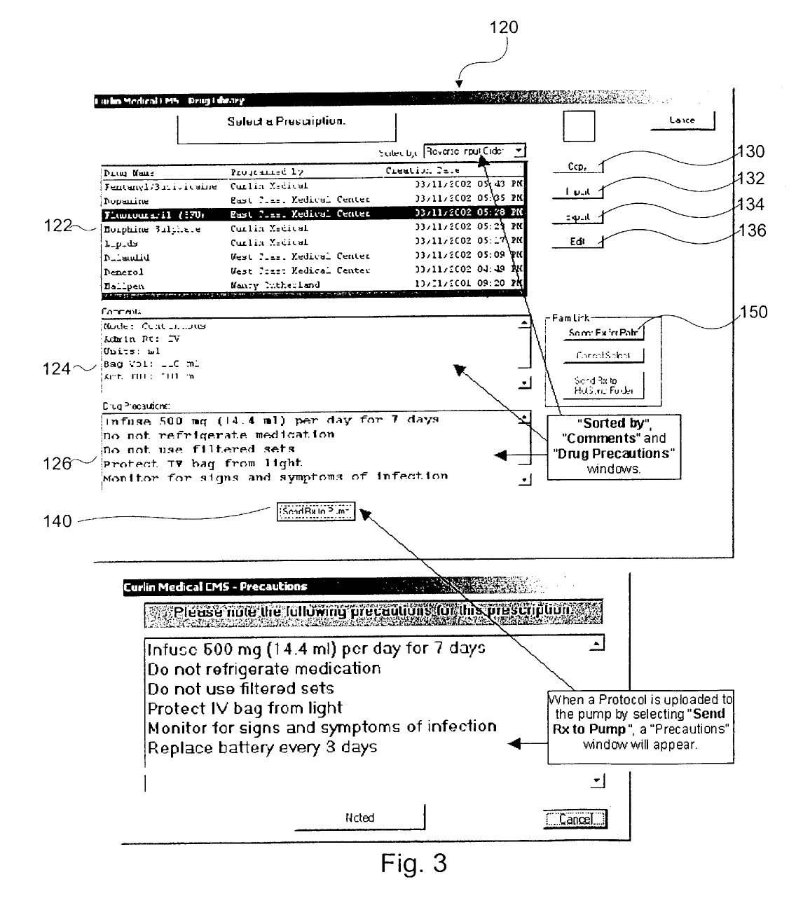

If the user presses the Drug Library button 102, an exemplary Drug Library Display 120 such as the one shown in FIG. 3 is displayed so that the user can view or edit information for the prescription that was entered during create Rx. In the example shown, there is a list of stored protocols 122 which are identified by a drug name, programmer and creation date. One of the stored protocols can be selected. Detailed information is then shown for the selected protocol. The detailed information includes comments 124 and drug precautions 126. The display includes controls, such as buttons, that allow the user to manipulate the data in the drug library.

In the exemplary embodiment, the user can press a Copy button 130 which allows the user to copy a protocol from the drug library to the patient library. When the copy button 130 is pressed, a window is displayed prompting for a patient's name. Entry and acceptance of a valid patient name causes the selected protocol to be copied to the patient library and stored under the entered patient's name.

Pressing an Import button 132 allows the user to import a protocol. This allows the user to store appropriately formatted files into the drug library. The files may be sent by another user.

Pressing an Export button 134 allows the user to export a selected protocol. The user can export the protocol to another user. In exemplary embodiments, the protocol is exported by sending it to the desired user via e-mail.

Pressing an Edit button 136 allows the user to edit an existing protocol. In exemplary embodiments, selection of the edit function causes two additional controls, e.g., buttons, to be displayed, namely, Delete and Save. The user can then edit the comments and/or precautions fields and save them by pressing the save button, if desired. In exemplary embodiments, the drug name, programmed by and creation date fields cannot be edited. The user may delete a protocol, if desired, by pressing the Delete button.

The user may also send the prescription to the pump by pressing a Send Rx to Pump button 140. Sending a prescription to the pump programs the pump with the prescription. Pressing the Send Rx to Pump button 140 causes the precaution window to display the precaution information for the protocol. The administrator of the prescription must review the precautions and indicate that the precautions have been reviewed by pressing the “Noted” button.

The user may also Send a Prescription to the Palm™ by pressing the Select Rx for Palm button 150. In exemplary embodiments, selection of this function saves selected protocols into a directory for transfer to a Palm™ device. A cable is connected from the computer to the Palm™ device. In exemplary embodiments, the user selects the prescription to be sent to a HotSync folder. The files in the HotSync folder can then be selected for transfer to the Palm™ device. In exemplary embodiments, all of the protocols being transferred to the Palm™ device are stored in one file, for example, a file named Patient.pdb. This file is then transferred to the Palm™ device. In exemplary embodiments, if there is an existing Patient.pdb file, it will be written over by the new file. Thus, the user must transfer all desired protocols to the Palm™ device as the previous protocols in an existing Patient.pdb file will be overwritten.

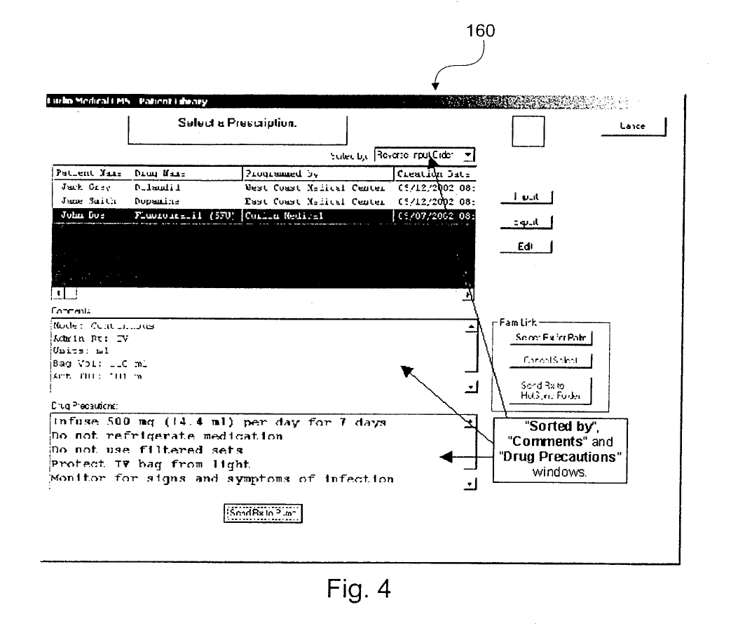

If the user presses the Patient Library button 104, a patient library display 160 is displayed. In exemplary embodiments, such as the one shown in FIG. 4, the patient library display 160 and functions (invoked by controls, such as buttons) are similar to those for the drug library. As with the drug library, the user can import, export or edit entries in the patient library. The user can send a prescription to the pump or transfer prescriptions to the Palm™ device.

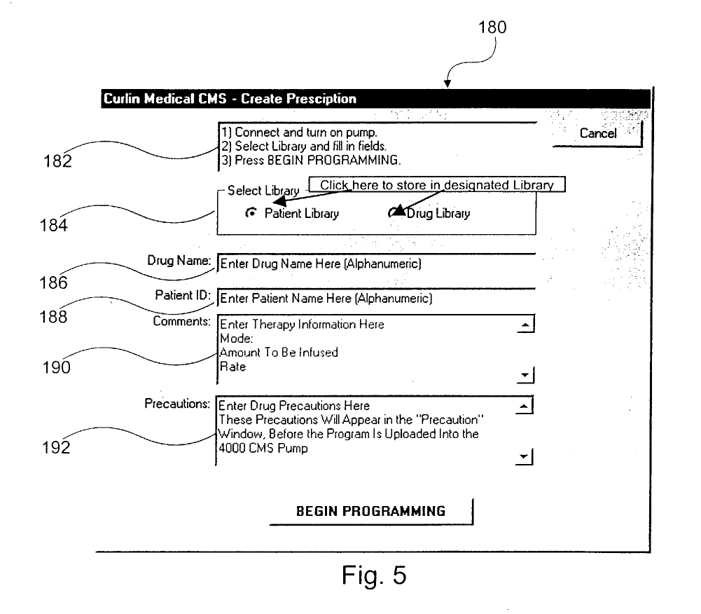

Creating a prescription allows the user to store prescription information in the drug library or the patient library. This information is uploaded from the pump. The user presses the Create Prescription button 106 from the main display window 100. A create prescription window 180 such as the one shown in FIG. 5 is displayed. The exemplary screen display shown in FIG. 5 provides the user with an instruction window 182 which tells the user to: (1) connect and turn on the pump; (2) select library and fill in fields; and (3) program the pump.

A cable is used to connect the pump, for example, Curlin™ Medical 4000 CMS pump 10 is connected to the PC 20, by inserting the cable in the serial port of the PC. The user selects the desired library 184, i.e., the drug library or the patient library, for storing the protocol to be uploaded from the pump. The user also enters a drug name 186, a patient ID 188, comments 190 and precautions 192.

The user then presses a Begin Programming button 194 and begins programming the pump. The pump is programmed the same as during stand-alone operation of the pump. For example, if the pump is a Curlin™ Medical 4000 Plus pump, the pump is programmed according to the directions for that particular pump. The user's manual for the Curlin™ Medical 4000 Plus pump is included as a compact disc appendix and is incorporated herein by reference.

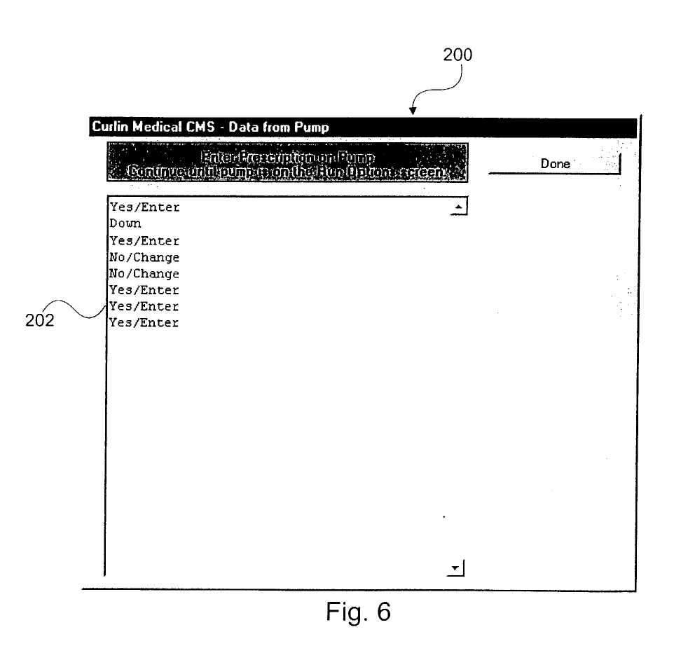

In exemplary embodiments, such as the one shown in FIG. 6, protocol information, including keystrokes that are used when programming the pump, is stored. For example, when a menu is displayed and the user scrolls down, “DOWN” is stored in the protocol file. Thus, when the information is uploaded to the pump, it is as if a user were using the keypad to enter the information directly into the pump except that the information is actually transmitted from the computer via the cable that connects the pump to the computer.

The computer stores history files. Peace of mind files include the latest programmed prescription.

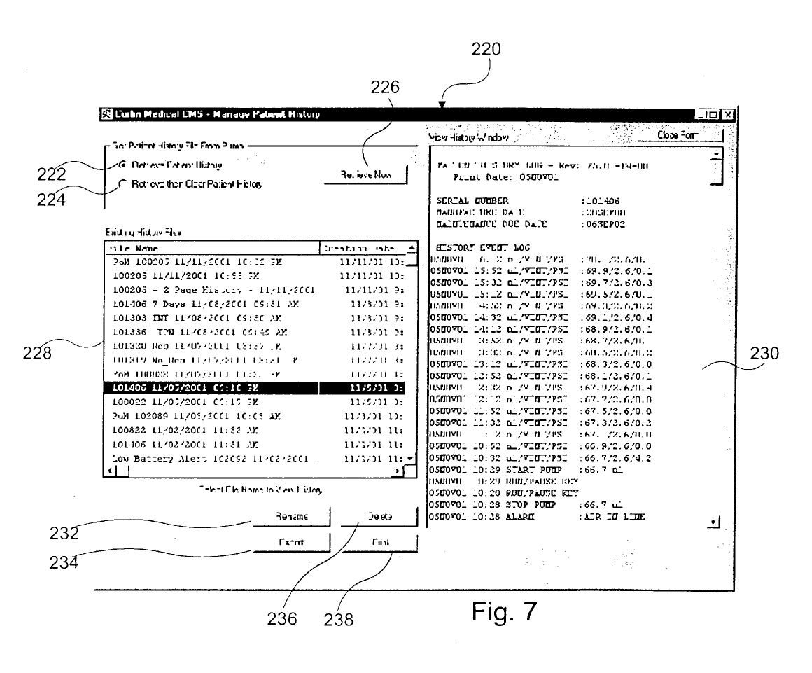

If the user presses the Manage History button 108, the manage history function is invoked and all of the information stored in the pump 10 is downloaded to the computer 20. A manage patient history display 220 such as the one shown in FIG. 7 is displayed. The user can either choose to retrieve the patient history 222 or to retrieve and then clear the patient history 224. If clear the patient history is selected, the history file will be deleted from the pump 10 after it is downloaded to the computer 20. After selecting one of these options, the user presses a Retrieve Now button 226 to retrieve the data from the pump. The names and creation dates of the downloaded history files are displayed in an existing history files window 228. The user can select a history file from the existing history file window 228. The data in the selected file is then displayed in a view history window 230. There are also controls (e.g., buttons) that allow the user to rename 232, export 234, delete 236 or print 238 a selected history file.

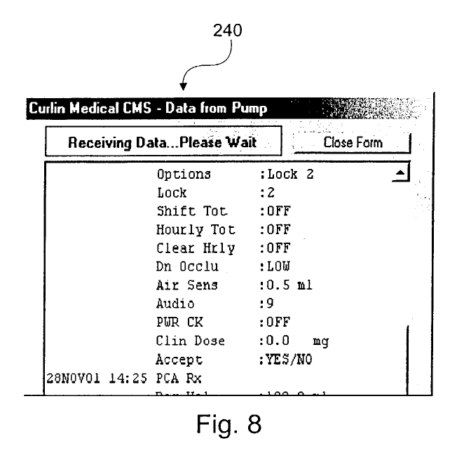

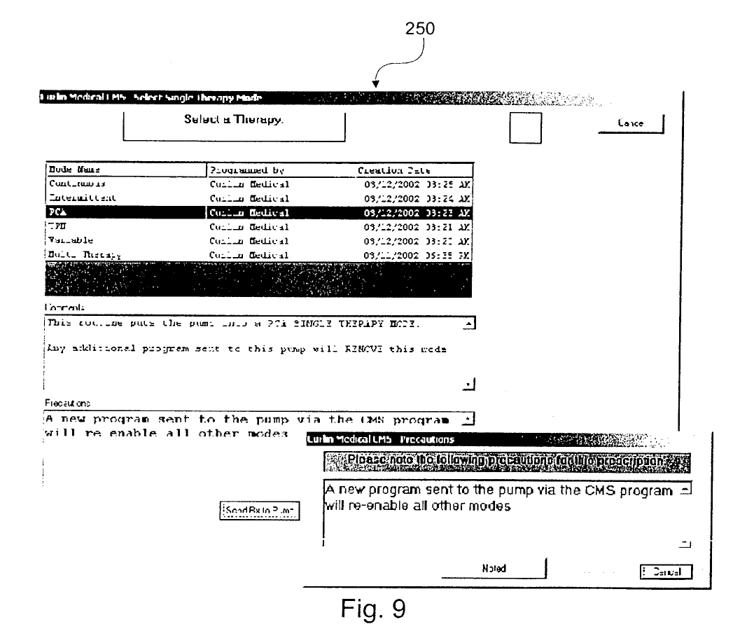

If the user presses the Peace of Mind button 112, a peace of mind function is invoked. The peace of mind function downloads and displays the most recently programmed therapy. This provides the clinician with proof (or peace of mind) that the therapy was uploaded into the pump. In exemplary embodiments, this feature downloads the most recent four (4) kilobytes of data from the pump history. An exemplary screen display 240 showing peace of mind data is shown in FIG. 8.

The present invention also allows the pump to be utilized as a single therapy device. When the user presses the Single Therapy button 112, all but one therapeutic mode on the pump are disabled. For example, some hospitals only need a single mode, such as PCA. This feature can be used when the pump is being manually programmed. In exemplary embodiments, the user can select any available therapeutic mode as the single mode therapy, for example, Continuous, Intermittent, PCA, TPN, or Variable. The pump can be removed from single therapy mode manually or by selecting Multi therapy.

Various maintenance activities may be performed on the pump using the present invention. For example, a current date/time may be entered and/or a maintenance date may be entered. The present invention may also be used to invoke calibration functions on the pump.

In exemplary embodiments, a palm computing device 30 may be used to perform a subset of the operations that can be performed by larger computing devices, such as a laptop computer 20. The Palm system can be used to program a pump 10 or to retrieve information from the pump. Protocols or prescriptions can be transferred to the PDA 30 from the PC 20 for bedside pump programming. Infusion information gathered by the pump 10 can be downloaded for later analysis.

In exemplary embodiments, clinical assessments and diagnostics can be performed. Preferably, these reports can be automatically generated. For example, the exemplary screen display 100 shown in FIG. 2 may include a control (not shown) for generating reports. Such a report could be printed or displayed. In exemplary embodiments, the reports are in a format that can be viewed using external off-the-shelf programs, such as Microsoft® Excel®.

One example clinical assessment and diagnostic that can be performed is the monitoring and analysis of patient side administration set line pressure. For example, a pump, such as the Curlin™ Medical 4000 pump manufactured by Curlin™ Technology of Huntington Beach, Calif. may be capable of monitoring patient side administration set line pressure. The monitored line pressure may be stored in a history file. The line pressure information can be retrieved from the history file and used to perform trend analysis.

Various catheters may be used with the pump. For example, the catheter may be a peripherally inserted central line catheter inserted up through the patient's arm or a subcutaneous port catheter used with a Huber™ needle. The pump infuses medication into the patient via the catheter.

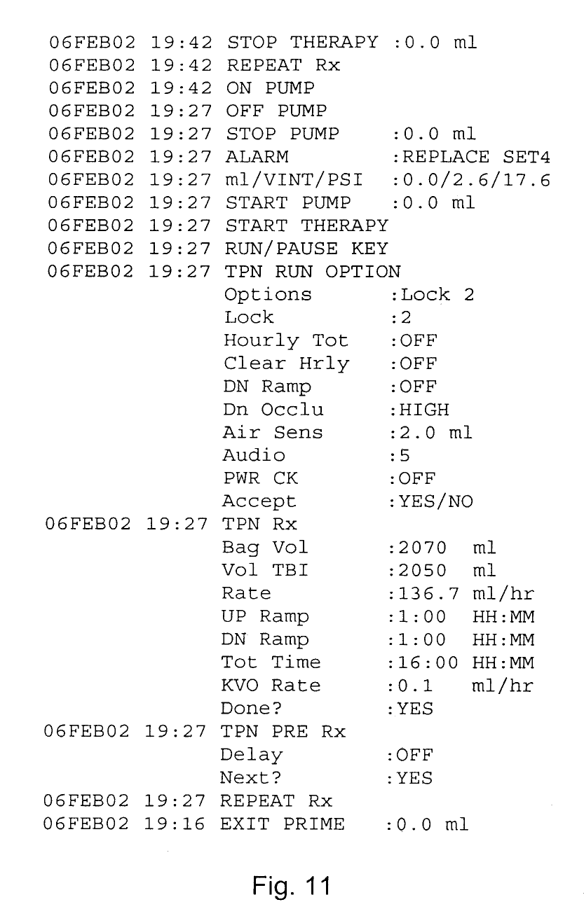

The internal pressure (in millimeters of mercury (mmHg)) is the infusion pressure leaving the pump and going into the patient. In exemplary embodiments, line pressure is continuously taken and is recorded every twenty minutes in the history file. An exemplary history file is included in the CD appendix and is titled “History File TPN Pressure SN102243.” A portion of this history file is shown in FIG. 11. The volume infused, voltage of the internal battery, and internal line pressure are recorded in the history file every twenty minutes.

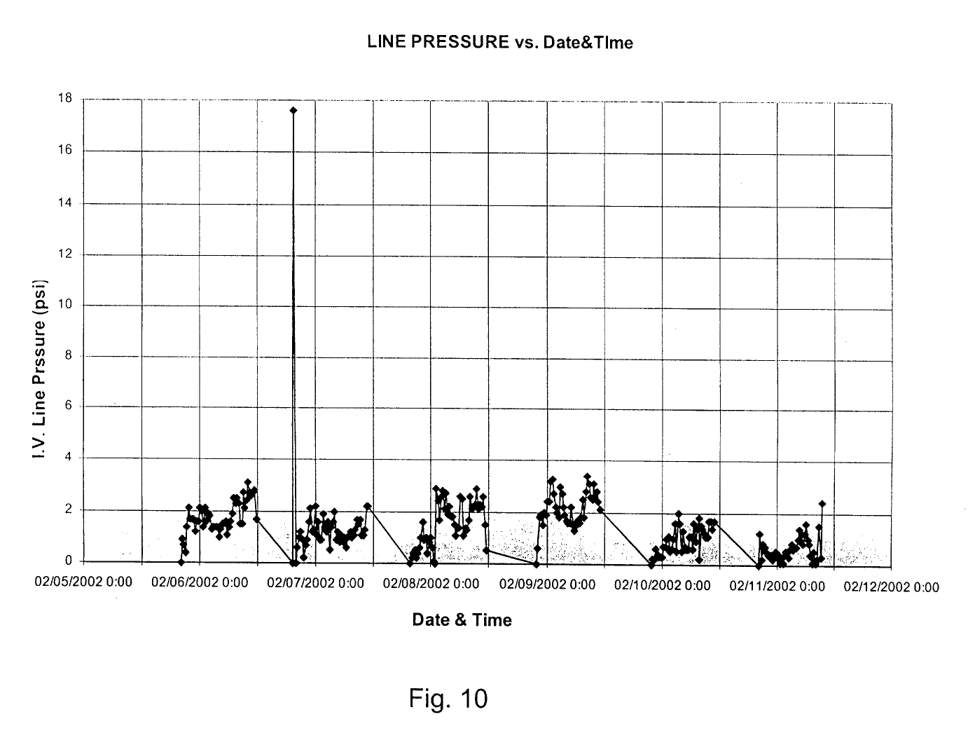

The data can be extracted from the history file to be analyzed. The data may be plotted. For example, FIG. 10 illustrates history data plotted from the file included in the CD appendix. This information can be displayed, printed and/or saved in a file. In exemplary embodiments, the data can be transmitted, for example via email. The saved files can be renamed and/or deleted.

The plot can be used to perform trend analysis. This trend analysis can be used to determine if pressure is building up. This build up of pressure indicates that there is a restriction of the flow of the medication into the patient. The analysis can also be used to assist in predicting when the catheter needs to be flushed or replaced.

In FIG. 10, after starting the infusion, the average pressure started to slowly increase on the third and fourth days. The pressure then dropped. This increase is likely due to gross sedimentation from the bag occluding the administration set filter. The pressure drop was likely because the bag was replaced.

Anomalies may also be seen in the graph. For example, there is a large spike in the pressure. This spike occurred immediately after starting infusion and then dropped prior to the next pressure reading. This indicates a problem that was quickly corrected. For example, a slide clamp may have been left in place which caused the spike. When the slide clamp was removed, the spike drop, i.e., the pressure returned to a normal or expected range value.

Total Parenteral Nutrition (TPN) patients are concerned about occlusion of the catheter. Such an occlusion can indicate the presence of an infection. Catheter pressure trend analysis as described above can be used to predict catheter performance and useful life. It can help to determine when a catheter needs to be flushed or replaced. This reduces the risk of infection to the patient due to catheter inclusion. A home healthcare professional can typically flush or replace a catheter. Trend analysis for determining when a catheter should be flushed or replaced may prevent unnecessary trips to the emergency room.

Another example of a diagnostic is the analysis of bolus requests. Hospitals typically configure a pump based on a single protocol. However, patients have different tolerances to pain. Patient Controlled Analgesia (PCA) therapy allows a patient to have some control over the amount of medication received. A prescription includes a basal amount. The patient may also be able to receive additional medication (a bolus) upon request. The bolus allows for a certain amount of medication to be administered above the basal amount. The patient has a mechanism, such as a button which is pressed, to request additional medication. Medication is not automatically administered for each request. The system is programmed to allow only a certain amount of additional medication within a given time period.

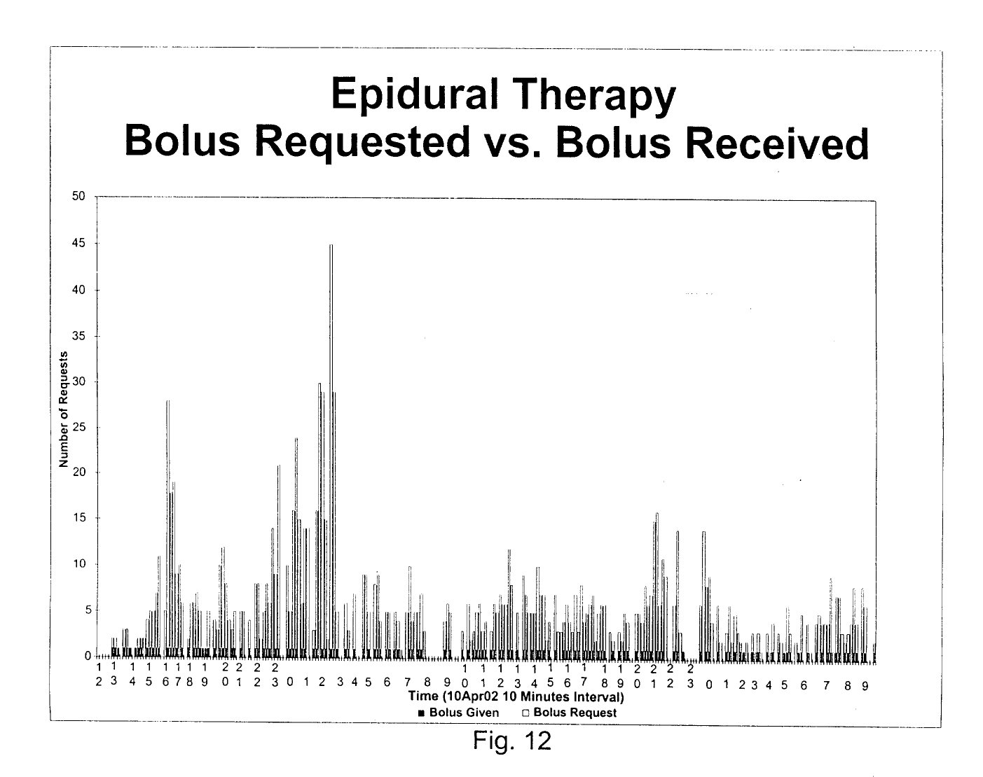

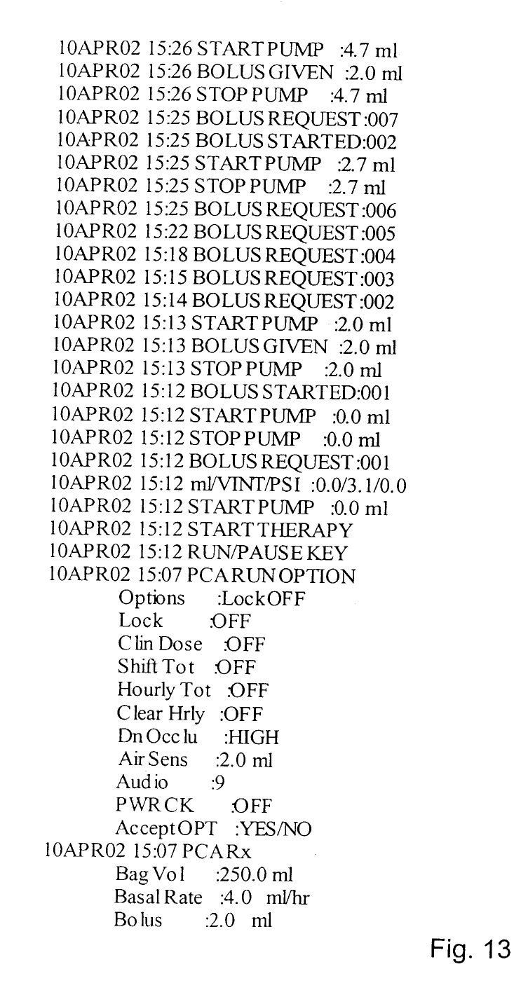

Bolus requests are recorded in the history file. Additional medication administered in request to the bolus request(s) is also stored in the history file. The bolus requests and medication administered in response to the bolus requests can be extracted from the history file. An exemplary history file is in the CD Appendix (in a file entitled “History File PCA Boluses UCLA_SN102436”). FIG. 13 shows an excerpt of this history file. The extracted information can be put in a graphical format such as that shown in FIG. 12. Trend analysis can be performed in order to determine a patient's tolerance for pain. This information can be displayed, printed and/or saved in a file. In exemplary embodiments, the data can be transmitted, for example via e-mail. The saved files can be renamed and/or deleted

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. A method for extracting data from history information received from a peristaltic pump, the method comprising:
receiving the history information from the peristaltic pump;
receiving a request for information to extract from the history file; and
extracting the requested information from the history file.

2. The method of claim 1, further comprising displaying the extracted information.

3. The method of claim 1, wherein the extracted information is displayed in a graphical format.

4. The method of claim 1, further comprising saving the extracted information in a file.

5. The method of claim 4, wherein the extracted information is saved in a graphical format.

6. The method of claim 4, further comprising:
receiving a request to delete the file; and
deleting the file.

7. The method of claim 4, further comprising:
receiving a request to rename the file; and
renaming the file.

8. The method of claim 1, further comprising printing the extracted information.

9. The method of claim 8, wherein the extracted information is printed in a graphical format.

10. The method of claim 1, further comprising transmitting the extracted information.

11. The method of claim 10, wherein the extracted information is transmitted in a graphical format.

12. The method of claim 10, wherein the extracted information is transmitted via e-mail.

13. The method of claim 1, wherein the requested information is patient side administration set line pressure information.

14. The method of claim 1, wherein the requested information is bolus request information.

15. The method of claim 14, wherein the bolus request information includes volume of medication administered in response to bolus requests.

16. A method for extracting bolus request data from history information received from a peristaltic pump, the method comprising:
receiving the history information from the peristaltic pump;
receiving a request to extract bolus request information from the history file; and
extracting the requested bolus request information from the history file.

17. The method of claim 16, further comprising displaying the extracted bolus request information.

18. The method of claim 16, wherein the extracted bolus request information is displayed in a graphical format.

19. The method of claim 16, wherein the bolus request information includes volume of medication administered in response to bolus requests.

20. A method for extracting patient side administration set line pressure data from history information received from a peristaltic pump, the method comprising:
receiving the history information from the peristaltic pump;
receiving a request for the patient side administration set line pressure information; and
extracting the requested patient side administration set line pressure information from the history file.

21. The method of claim 20, further comprising displaying the extracted patient side administration set line pressure information.

22. The method of claim 20, wherein the extracted patient side administration set line pressure information is displayed in a graphical format.
